(19) United States
(12) Patent Application Publication
(10) Pub. No.: US 2023/0146570 A1
(43) Pub. Date: May 11, 2023

(54) PCR RAPID DETECTION DEVICE AND METHOD THEREOF

(71) Applicant: INDUSTRIAL TECHNOLOGY RESEARCH INSTITUTE, Hsinchu (TW)

(72) Inventors: Ta-Wei LIN, Zhubei City (TW); Yung-Pin LEE, New Taipei City (TW); Wei-Lun LIANG, Kaohsiung City (TW)

(73) Assignee: INDUSTRIAL TECHNOLOGY RESEARCH INSTITUTE, Hsinchu (TW)

(21) Appl. No.: 17/550,771

(22) Filed: Dec. 14, 2021

(30) Foreign Application Priority Data
Nov. 8, 2021 (TW) 110141503

(51) Int. Cl. B01L 3/00; B01L 7/00; C12Q 1/686

(52) U.S. Cl. CPC B01L 3/502715; B01L 7/52; B01L 3/502784; C12Q 1/686; B01L 3/502707; B01L 2200/16; B01L 2200/12; B01L 2300/0663; B01L 2300/0883; B01L 2300/0887; B01L 2400/043

(57) ABSTRACT

A rapid PCR detection device includes a body, a disposable microfluidic unit, a magnetron micro-fluid unit, a linear actuator, a PCR thermal cycling unit and an image recognition unit. The microfluidic unit is made of a transparent material, wherein a transparent film is arranged in the middle of the microfluidic channel and has at least one hole for a micro-fluid to flow in the microfluidic channel. The magnetron micro-fluid unit drives the micro-fluid, so that the micro-fluid is divided into a plurality of droplets each guided to a lower layer of the microfluidic channel. The linear actuator drives the disposable microfluidic unit to an amplification zone. The PCR thermal cycling unit performs PCR thermal cycling in the amplification zone. The image recognition unit illuminates the droplets with a fluorescent light and determines the number of DNA fragments in the droplets according to the detected fluorescent intensity.

This application claims the benefit of Taiwan application Serial No. 110141503, filed Nov. 8, 2021, the subject matter of which is incorporated herein by reference.

### TECHNICAL FIELD

The disclosure relates in general to a detection device, and more particularly to a rapid polymerase chain reaction (PCR) detection device and a method thereof.

### BACKGROUND

Polymerase chain reaction (PCR) is an enzyme driven process for amplifying the deoxyribonucleic acid (DNA) fragments in vitro. Through denaturation, annealing and elongation, PCR can duplicate millions of DNA fragments. In the denaturation step, double helix DNA is denaturized into single helix DNA at a high temperature (90-95° C.), then the single helix DNA is used as a duplication template. In the annealing step, when the temperature drops to a suitable level, the primers can be annealed to correct positions of the target gene. In the elongation step, the temperature is adjusted to 72° C., and magnesium ions are used as enzyme cofactor, so that DNA polymerase can be synthesized as another strand of DNA fragment according to the code of the duplication template. By repeating the above three steps continuously, a small volume of DNA fragment can be duplicated to a large volume.

Although the real-time nucleic acid PCR (RT-PCR) test is simple and has excellent performance in amplification, absolute quantitative result still cannot be obtained. On the other hand, digital nucleic acid PCR (dPCR) test can perform quantitative analysis using direct counting method but requires human intervention at the transition between different stages of the testing process, not only deteriorating testing efficiency but also increasing cost and the operating complexity of device and adding risk to the operator.

### SUMMARY

The disclosure is directed to a rapid PCR detection device whose disposable microfluidic unit, magnetron micro-fluid unit, linear actuator, PCR thermal cycling unit and image recognition unit are integrated in a body, so that integrated rapid testing can be achieved.

According to one embodiment of the present disclosure, a rapid PCR detection device is provided. The device includes a body, a disposable microfluidic unit, a magnetron micro-fluid unit, a linear actuator, a PCR thermal cycling unit and an image recognition unit. The microfluidic unit is made of a transparent material, wherein a transparent film is arranged in the middle of the microfluidic channel and has at least one hole for a micro-fluid to flow in the microfluidic channel. The magnetron micro-fluid unit is used to drive the micro-fluid, so that the micro-fluid is divided into a plurality of droplets guided to the lower layer of the microfluidic channel. The linear actuator is used to drive the disposable microfluidic unit to an amplification zone of the body. The PCR thermal cycling unit performs PCR thermal cycling in the amplification zone. The image recognition unit illuminates the droplets with a fluorescent light and determines the number of DNA fragments in the droplets according to the detected fluorescent intensity.

According to another embodiment of the present disclosure, a rapid PCR detection method is provided. The method includes the following steps. A micro-fluid is placed in a disposable microfluidic unit, wherein a transparent film is arranged in the middle of the microfluidic channel and has at least one hole, and the micro-fluid flows in an upper layer of the microfluidic channel. A magnet is controlled to move under the microfluidic channel and drive the micro-fluid, so that the micro-fluid is divided into a plurality of droplets guided to the lower layer of the microfluidic channel. The disposable microfluidic unit is driven to an amplification zone of a body. A PCR thermal cycling is performed in the amplification zone. The droplets are illuminated with a fluorescent light and the number of DNA fragments in the droplets is determined according to the detected fluorescent intensity.

The above and other aspects of the disclosure will become better understood with regard to the following detailed description of the preferred but non-limiting embodiment(s). The following description is made with reference to the accompanying drawings.

### BRIEF DESCRIPTION OF THE DRAWINGS

FIG. 1 is a block diagram of a rapid PCR detection device according to an embodiment of the present disclosure;

FIGS. 2A and 2B respectively are schematic diagrams of internal configuration of a rapid PCR detection device according to an embodiment of the present disclosure;

FIGS. 3A and 3B respectively are an explosion diagram and an assembly diagram of a microfluidic channel according to an embodiment of the present disclosure.

FIGS. 4A-4C respectively are schematic diagrams of droplet division, PCR amplification and image recognition performed in a detection method according to an embodiment of the present disclosure;

FIG. 5A is a schematic diagram of a magnetron micro-fluid unit according to an embodiment of the present disclosure;

FIG. 5B is a schematic diagram of droplets moved to a predetermined position along a magnetic induction track according to an embodiment of the present disclosure;

FIG. 6 is a schematic diagram of a drive circuit according to an embodiment of the present disclosure;

FIG. 7 is a flowchart of a rapid PCR detection method according to an embodiment of the present disclosure.

### DETAILED DESCRIPTION

Technical solutions for the embodiments of the present disclosure are clearly and thoroughly disclosed with accompanying drawings. However, the embodiments disclosed below are only some rather than all of the embodiments of the present disclosure. All embodiments obtained by anyone ordinarily skilled in the technology field of the disclosure according to the disclosed embodiments of the present disclosure are within the scope of protection of the present disclosure if the obtained embodiments lack innovative labor.

The disclosed features, structures or characteristics can be combined in one or more embodiments by any suitable way. In the following disclosure, many detailed descriptions are provided for the embodiments of the present disclosure to be better and fully understood. However, anyone ordinarily skilled in the technology field of the disclosure will understand that technical solution for implementing the present disclosure also can dispense with one or more of the details disclosed below or can be implemented using other methods, devices, or steps. In some circumstances, generally known methods, devices, implementations, or operations of the technical solution capable of implementing the present disclosure are not necessarily illustrated or disclosed in detail lest the aspects of the present disclosure might be distracted.

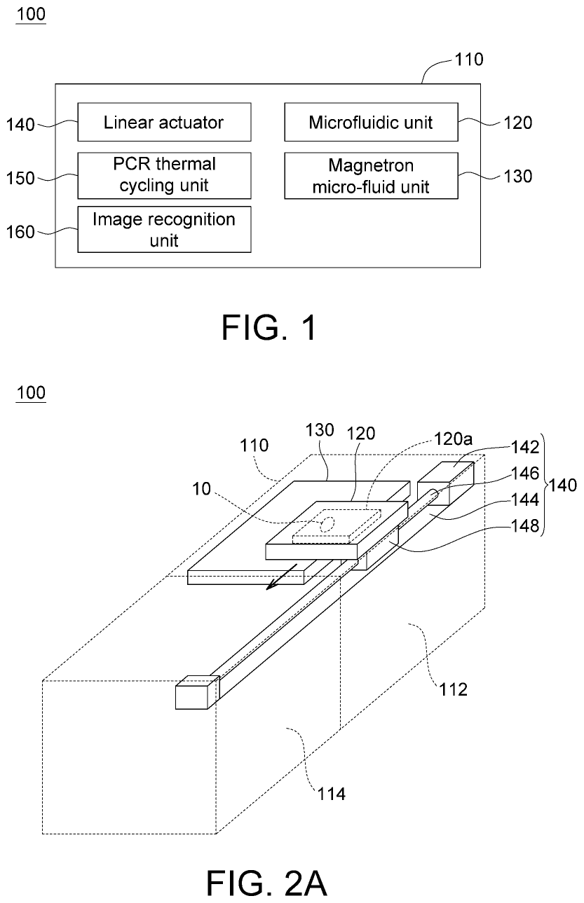

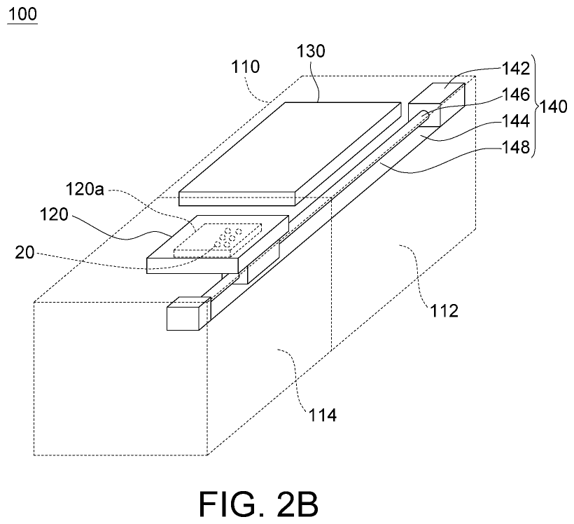

Refer to FIG. 1 and FIGS. 2A and 2B. FIG. 1 is a block diagram of a rapid PCR detection device 100 according to an embodiment of the present disclosure. FIGS. 2A and 2B respectively are schematic diagrams of internal configuration of a rapid PCR detection device 100 according to an embodiment of the present disclosure. The rapid PCR detection device 100 of the present embodiment includes a microfluidic unit 120, a magnetron micro-fluid unit 130, a linear actuator 140, a PCR thermal cycling unit 150 and an image recognition unit 160, which are disposed in a body 110 and form an integrated rapid testing platform.

The body 110 is an operating platform on which the user can input setting values, such as thermal cycling temperature value and thermal cycling time, analyze image parameters, detect fluorescent intensity, calculate the number of DNA fragments, and output the detected values. All of the above testing steps are completed in one single body 110 instead of being allocated to different testing devices, hence avoiding human intervention which would occur at the transition between different stages of the testing process.

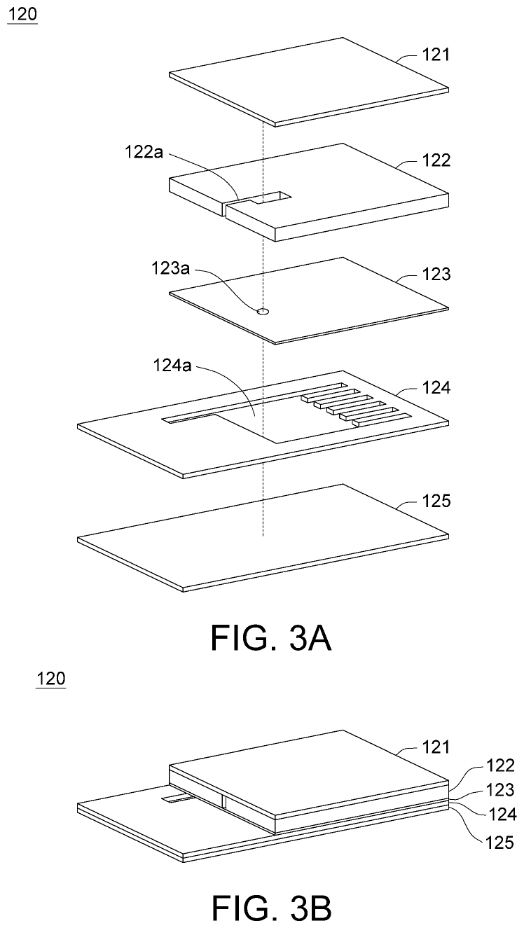

Refer to FIGS. 2A and 2B. The interior of the body 110 is divided into a first area 112 and a second area 114. The magnetron micro-fluid unit 130 is located in the first area 112; the PCR thermal cycling unit 150 and the image recognition unit 160 are located in the second area 114. The first area 112 of the body 110 for droplet division; the second area 114 is for PCR amplification and fluorescence detection. The linear actuator 140 is movably disposed between the first area 112 and the second area 114. The disposable microfluidic unit 120 is driven by the linear actuator 140 to reciprocally move between the first area 112 and the second area 114. The interior of the microfluidic unit 120 has two layers of microfluidic channel 120a, that is, an upper layer and a lower layer of the microfluidic channel 122a and 124a as indicated in FIG. 3A.

In an embodiment, the linear actuator 140 includes a motor 142, a linear slide 144 and a ball screw 146. The ball screw 146 is rotated by a torque provided by the motor 142. The slider 148 on the linear slide 144 and the adapter (not illustrated) on the ball screw 146 are integrally coupled in one piece and move along the linear slide 144. The disposable microfluidic unit 120 is disposed on the slider 148 and can slide to an amplification zone (the amplification zone is located with the second area 114) to perform digital nucleic acid PCR (dPCR) test.

Referring to FIGS. 3A and 3B, an explosion diagram and an assembly diagram of a microfluidic channel according to an embodiment of the present disclosure are respectively shown. The microfluidic unit 120 is made of a transparent material (such as acrylic, PMMA or PDMS) and formed of layered plates (such as 5 layers), wherein the plates are such as a cover 121, an upper plate 122, an intermediate plate 123, a lower plate 124 and a bottom plate 125. The upper plate 122 and the lower plate 124 respectively have microfluidic channels 122a and 124a. The intermediate plate 123 is formed of a transparent film, which has at least one hole 123a. Each hole 123a is interconnected between the microfluidic channel 122a of the upper plate 122 and the microfluidic channel 124a of the lower plate 124 for a micro-fluid 10 to flow in the microfluidic channel. The micro-fluid 10 moves to the microfluidic channel 124a of the lower plate 124 from the microfluidic channel 122a of the upper plate 122 through the hole 123a arranged in the intermediate plate 123.

In comparison to the conventional microfluidic silicon chip with high cost, the microfluidic unit 120 incurs lower cost and is easier to manufacture. Furthermore, the microfluidic channel can be customized according to customer needs and can be disposed after one time of use, hence reducing the probability of the microfluidic channel being polluted by the residuals of the DNA tester. Besides, the microfluidic unit 120 can completely seal the micro-fluid 10 and divide into the droplets 20 through magnetron. Hence, the transition between different stages of the testing process does not require human intervention, the risk of human contact can be reduced, and the automation of nucleic acid PCR test can be implemented.

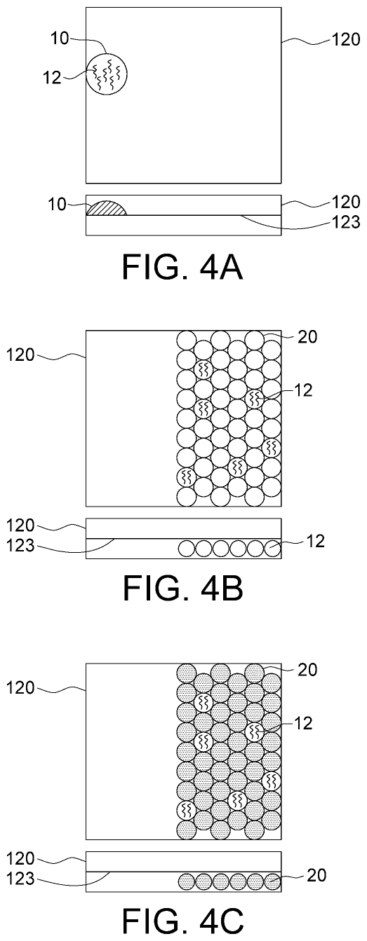

Refer to FIGS. 4A-4C, a top view and a side view of droplet division, PCR amplification and image recognition performed in a detection method according to an embodiment of the present disclosure are respectively shown. Refer to FIGS. 2A, 3A and 5A. As indicated in FIG. 4A, when the microfluidic unit 120 is located in the first area 112, the micro-fluid 10 formed of the to-be-tested DNA tester, ferrofluid, and PCR fluorescent dye is placed in the microfluidic channel 122a of the upper plate 122. Then, the permanent magnet 132 is driven by an electromagnetic force generated by the magnetron micro-fluid unit 130, and the micro-fluid 10 is driven by the permanent magnet 132 to move in the microfluidic channel 122a of the upper plate 122, and the micro-fluid 10, after passing through the hole 123a of the intermediate plate 123, is divided to form a droplet 20. The said droplet division process is repeated to obtain a plurality of droplets 20.

Refer to FIGS. 2A, 3A, 4B and 5A. In FIG. 4B, after droplet division is completed, the droplet 20 is driven by the permanent magnet 132 and sequentially moved to the microfluidic channel 124a of the lower plate 124 for subsequent PCR amplification. Refer to FIGS. 2B, 4B and 5A. In FIG. 4B, the linear actuator 140 drives the microfluidic unit 120 to move to the second area 114 from the first area 112, and the PCR thermal cycling unit 150 performs PCR thermal cycling in the second area 114. In the PCR thermal cycling process, double helix DNA is denaturized into single helix DNA at a high temperature. Then, the single helix DNA is used as a duplication template. When the temperature drops to a suitable level, the primers can find the two ends of a target gene fragment and be annealed thereto. Polymerase can correctly add 4 nucleic acid materials (dNTPs:dATP, dGTP, dCTP, dTTP) to the fragment one by one according to the code of the DNA template to form a new strand of DNA fragment. After the PCR thermal cycling process is repeated for a predetermined number of repeats, a large number of duplicated DNA fragments will be obtained. In the present embodiment, the predetermined number of repeats is 30-40 times, but the present disclosure is not limited thereto.

Refer to FIGS. 2B and 4C. In FIG. 4C, the image recognition unit 160 illuminates the droplets with a fluorescent light, 20, and determines, according to the detected fluorescent intensity, the number of DNA fragments 12 duplicated in the droplets 20 during the PCR thermal cycling process. Since the number of DNA fragments 12 duplicated in each droplet 20 is not the same, the image recognition unit 160 can calculate the fluorescent intensity of each droplet 20 according to the luminous flux of the image sensor corresponding to each droplet 20 or calculate the fluorescent intensity of each droplet 20 according to the luminous flux of different pixels in one single image sensor. Through image recognition, the number of DNA fragments in each droplet 20 can be determined to complete quantitative analysis.

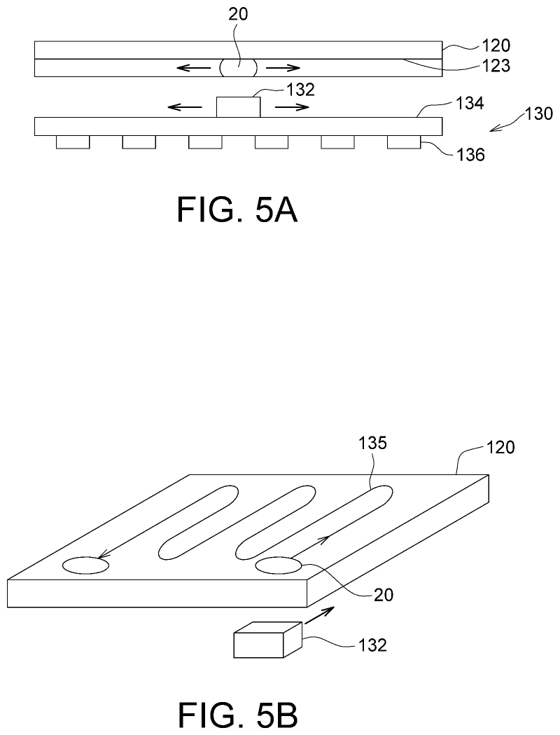

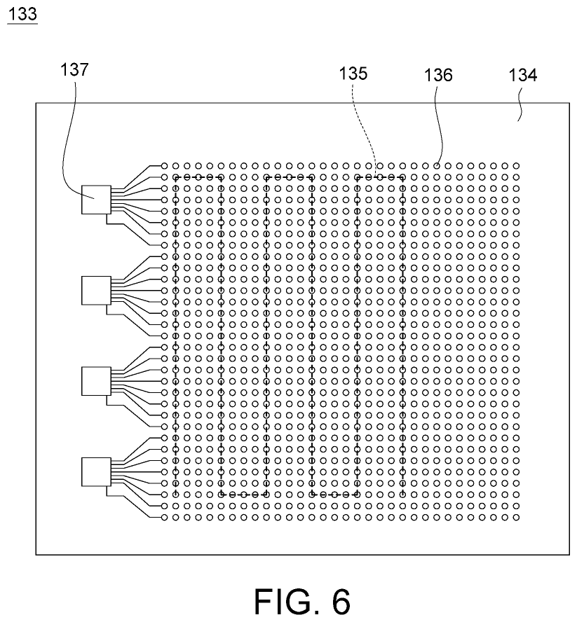

Refer to FIGS. 5A, 5B and 6. FIG. 5A is a schematic diagram of a magnetron micro-fluid unit 130 according to an embodiment of the present disclosure. FIG. 6 is a schematic diagram of a drive circuit 133 according to an embodiment of the present disclosure. The magnetron micro-fluid unit 130 includes a magnet 132, a drive circuit 133 and a magnetic induction track 137. The drive circuit 133 includes a printed circuit board 134, a drive element 137 and a plurality of electromagnetic coils 136 disposed on the printed circuit board 134. The magnet 132 is disposed on the top of the printed circuit board 134. The electromagnetic coils 136 is disposed under the printed circuit board 134. The drive element 137 provides a drive current to a portion of electromagnetic coils 136 sequentially, so that the electrified electromagnetic coils 136 can form a magnetic induction track 135 of any shape to generate a magnetic force. The magnetic force generated by the drive circuit 133 drives the magnet 132 to move on the printed circuit board 134. As indicated in FIG. 6, a plurality of electromagnetic coils 136 are arranged as an array on the printed circuit board 134. Each electromagnetic coil 136 can be powered by an independent power supply switch, so that the electrified electromagnetic coils 136 can be sequentially turned on to form a magnetic induction track 135 according to the predetermined moving path of the magnet 132 and further drives the magnet 132 to move on the printed circuit board 134.

Referring to FIG. 5B, a schematic diagram of a droplet 20 moved to a predetermined position along a magnetic induction track 135 according to an embodiment of the present disclosure is shown. The shape of the magnetic induction track 135 is S shape, E shape, F shape, T shape or a combination thereof, and is not subjected to specific restrictions in the present embodiment. The sequential ON/OFF time of the electromagnetic coils 136 can be predetermined, so that the droplet 20 can move to the predetermined position at the terminal of the magnetic induction track 135 and stop there. When the next droplet is desired to move, the predetermined position of the next droplet 20 can be controlled by changing the power-on location of the electromagnetic coils 136 and the said process is repeated until the allocation of all droplets 20 is completed.

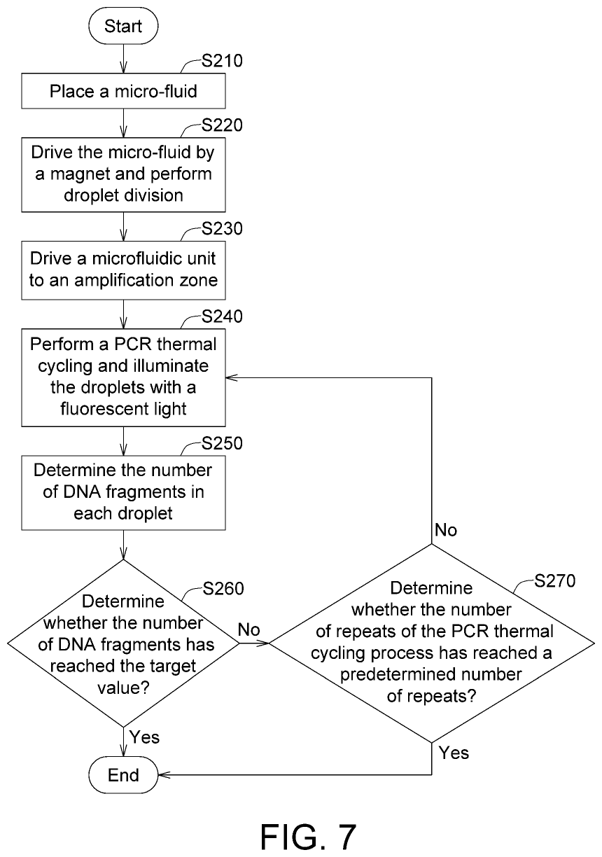

Referring to FIG. 7, a flowchart of a rapid PCR detection method according to an embodiment of the present disclosure is shown. Firstly, in step S210, a micro-fluid 10 is placed in a microfluidic unit 120, and a transparent film is arranged in the middle of the microfluidic channel, wherein the transparent film has at least one hole 123a and the micro-fluid 10 can flow in the upper layer of the microfluidic channel. In step S220, a magnet 132 is controlled to move under the microfluidic channel, and the micro-fluid 10 is driven by the magnet 132, so that the micro-fluid 10 is attracted by the magnet 132 and therefore is divided into a plurality of droplets 20 sequentially and each of the droplets 20 is guided to the lower layer of the microfluidic channel. In step S230, the microfluidic unit 120 is driven to an amplification zone of the body 110 (the amplification zone is within the second area 114). In step S240, PCR thermal cycling is performed in the amplification zone, and the droplets 20 are illuminated with a fluorescent light. In step S250, the number of DNA fragments in each of the droplets 20 is determined according to the detected fluorescent intensity. In step S260, if the number of DNA fragments has reached the target value, the entire testing process is completed. In step S270, if the number of DNA fragments has not reached the target value, and the number of repeats of the PCR thermal cycling process does not reach a predetermined number of repeats, the method returns to step S240 to continue the PCR thermal cycling process. If the number of DNA fragments has not reached the target value, and the PCR thermal cycling process has reached the predetermined number of repeats, the testing method terminates. In the present embodiment, the predetermined number of repeats is 30-40, but the present disclosure is not limited thereto.

The above disclosure shows that in actual operation, the rapid PCR detection device and method disclosed in the above embodiments of the present disclosure can be used in the quantitative analysis of digital nucleic acid PCR (dPCR) test and has the advantages of high degree of automation, lower cost and lower pollution risk. Furthermore, since droplet division, PCR thermal cycling and fluorescence detection can be completed in one single body instead of being allocated to different testing devices, human intervention which would occur at the transition between different stages of the testing process can be avoided. Additionally, the PCR testing process of the present embodiment can speed up PCR and reduce the testing time to be within 1.5 hours, so that the testing capacity of each testing center can be increased.

While the disclosure has been described by way of example and in terms of the preferred embodiment(s), it is to be understood that the disclosure is not limited thereto. On the contrary, it is intended to cover various modifications and similar arrangements and procedures, and the scope of the appended claims therefore should be accorded the broadest interpretation so as to encompass all such modifications and similar arrangements and procedures.

What is claimed is:

1. A rapid PCR detection device, comprising:
a body;
a disposable microfluidic unit made of a transparent material, wherein a transparent film is arranged in a middle of the microfluidic channel and has at least one hole for a micro-fluid to flow in the microfluidic channel;
a magnetron micro-fluid unit used to drive the micro-fluid, so that the micro-fluid is divided into a plurality of droplets and each of the drops is guided to a lower layer of the microfluidic channel;
a linear actuator used to move the disposable microfluidic unit to an amplification zone of the body;
a PCR thermal cycling unit used to perform a PCR thermal cycling on the droplets in the amplification zone; and
an image recognition unit used to illuminate the droplets with a fluorescent light and determine a number of DNA fragments in the droplets according to a detected fluorescent intensity.

2. The device according to claim 1 used in a quantitative analysis of digital nucleic acid PCR (dPCR) test.

3. The device according to claim 1, wherein droplet division, PCR thermal cycling and fluorescence detection are all performed in the single body.

4. The device according to claim 1, wherein an interior of the body comprises a first area and a second area, the magnetron micro-fluid unit is located in the first area, the PCR thermal cycling unit and the image recognition unit are located in the second area.

5. The device according to claim 4, wherein the linear actuator is movably disposed between the first area and the second area, the disposable microfluidic unit is driven by the linear actuator to move between the first area and the second area.

6. The device according to claim 1, wherein the disposable microfluidic unit comprises a cover, an upper plate, an intermediate plate, a lower plate and a bottom plate arranged in a top down manner, the upper plate and the lower plate respectively have a first microfluidic channel and a second microfluidic channel, the intermediate plate is the transparent film, and the hole is interconnected between the first microfluidic channel of the upper plate and the second microfluidic channel of the lower plate.

7. The device according to claim 1, wherein the magnetron micro-fluid unit comprises a magnet, a magnetic induction track and a drive circuit; the drive circuit controls the magnet to move under the microfluidic channel, and the magnet drives the micro-fluid to move along the magnetic induction track.

8. The device according to claim 7, wherein the drive circuit comprises a printed circuit board, a drive element and a plurality of electromagnetic coils disposed on the printed circuit board, the magnet is disposed on top of the printed circuit board; the electromagnetic coils is disposed under the printed circuit board; the drive element provides a drive current to a portion of the electromagnetic coils.

9. The device according to claim 8, wherein a portion of electrified electromagnetic coils forms the magnetic induction track corresponding to a predetermined moving path of the magnet.

10. The device according to claim 1, wherein the linear actuator comprises a motor, a linear slide and a ball screw, the motor provides a torque to rotate the ball screw, a slider on the linear slide and an adapter on the ball screw are integrally coupled in one piece and move along the linear slide.

11. A rapid PCR detection method used in a body, wherein the method comprises:
placing a micro-fluid in a disposable microfluidic unit, wherein a transparent film is arranged in a middle of a microfluidic channel, the transparent film has at least one hole for the micro-fluid to flow in the microfluidic channel;
controlling a magnet to move under the microfluidic channel and driving the micro-fluid by the magnet, so that the micro-fluid is divided into a plurality of droplets and each of the droplets is guided to a lower layer of the microfluidic channel; and
moving the disposable microfluidic unit to an amplification zone of the body;
performing a PCR thermal cycling in the amplification zone to adjust a temperature of the droplets; and
illuminating the droplets with a fluorescent light and determining a number of DNA fragments in the droplets according to a detected fluorescent intensity.

12. The method according to claim 11, wherein the method is used in a quantitative analysis of digital nucleic acid PCR (dPCR) test.

13. The method according to claim 11, wherein droplet division, PCR thermal cycling and fluorescence detection are all performed in the body.

14. The method according to claim 11, wherein the droplet division is located in a first area of the body, the PCR thermal cycling and the fluorescence detection are located in a second area of the body, and the disposable microfluidic unit is movable between the first area and the second area.

15. The method according to claim 11, wherein the hole is interconnected between a first microfluidic channel of a upper plate and a second microfluidic channel of a lower plate.

16. The method according to claim 11, wherein the magnet drives the micro-fluid to move along a magnetic induction track, a portion of electrified electromagnetic coils forms the magnetic induction track corresponding to a predetermined moving path of the magnet.
